(19) United States
(12) Patent Application Publication
(10) Pub. No.: US 2016/0177342 A1
(43) Pub. Date: Jun. 23, 2016

(54) SEED MEDIUM FOR THE CULTIVATION OF YEAST CELLS AND USES OF THE SAME

(71) Applicant: Far Eastern New Century Corporation, Taipei City (TW)

(72) Inventor: To-Chun Chao, Taipei City (TW)

(21) Appl. No.: 14/805,873

(22) Filed: Jul. 22, 2015

(30) Foreign Application Priority Data
Dec. 22, 2014 (TW) 103144784

(51) Int. Cl. C12P 7/06; C12N 1/18; C12N 1/16

(52) U.S. Cl. CPC C12P 7/06; C12N 1/16; C12N 1/18

(57) ABSTRACT

Disclosed herein is a seed medium for the cultivation of a yeast cell capable of yielding ethanol by consumption of glucose and xylose, consisting essentially of molasses, corn steep liquor and water, wherein based on the total volume of the seed medium, the molasses has a concentration ranging from 1.0 to 6.0% (v/v), and the corn steep liquor has a concentration ranging from 4.0 to 8.0% (v/v), the concentration of the molasses being less than that of the corn steep liquor. A seed culture of a yeast cell obtained using the seed medium and a method for producing ethanol from a biomass using the seed culture are also disclosed.

### CROSS-REFERENCE TO RELATED APPLICATIONS

This application claims priority of Taiwanese Application No. 103144784, filed on Dec. 22, 2014.

### BACKGROUND OF THE INVENTION

1. Field of the Invention

The disclosure relates to a seed medium for the cultivation of a yeast cell capable of yielding ethanol by consumption of glucose and xylose, more particularly to a seed medium consisting essentially of molasses, corn steep liquor and water, which can be used in cultivation of a yeast cell so that the yeast cell is effective in producing ethanol by utilization and metabolism of a biomass under stress conditions.

2. Background Information

A cellulosic biomass is a kind of renewable energy resource and can be abundantly produced through industry and agroforestry operations. Chemical or biological methods of converting the cellulosic biomass to biomass energy (i.e. cellulosic ethanol) have been widely studied and investigated by researchers. Due to the merits of having low energy consumption and being environment-friendly, biological methods have been the focus of research as compared to chemical methods.

In the process of producing ethanol from a cellulosic biomass, it is necessary to first hydrolyze the cellulosic biomass to obtain a cellulosic hydrolysate. The cellulosic hydrolysate usually contains reducing sugars and fermentation inhibitors, e.g., acetic acid, furfural, hydroxymethyl furfural (HMF), phenolic compounds, etc., generated by degradation of hemicellulose and reducing sugars during the process.

Reportedly, ethanol yield may be increased by subjecting Saccharomyces cerevisiae to gene modification. However, the fermentation inhibitors contained in the cellulosic hydrolysate may inhibit growth and fermentation of Saccharomyces cerevisiae, thereby adversely affecting the utilization rate of the reducing sugars and the ethanol yield. For example, E. Casey et al. found that the presence of acetic acid in a YEP fermentation medium containing glucose and xylose could significantly reduce ethanol yield and glucose and xylose consumption rates (especially xylose consumption rate). E. Casey et al. also disclosed that the inhibitory effect of acetic acid could be reduced by continuously adding ammonium hydroxide in the fermentation medium during fermentation (E. Casey et al. (2010), FEMS Yeast Research, 10:385-393). However, addition of ammonium hydroxide would increase the pH value of the fermentation medium, thereby increasing the risk of contamination and raising fermentation costs. Accordingly, much effort has been put into providing a fermentation method capable of decreasing undesired effects caused by fermentation inhibitors (particularly acetic acid) and increasing the xylose utilization rate and the ethanol yield.

Many by-products in the food industry contain abundant nutrient sources for microorganisms and thus exhibit recycling characteristics. It has been reported that the by-products can be used to cultivate microorganisms so as to produce valuable products. Molasses is a by-product in the sugar manufacturing industry, contains large amounts of sugar (including sucrose, glucose, fructose, etc.) and water, and can be used as carbon sources in media and additives of food or feed. Molasses may be classified into cane molasses (having a total sugar content of about 48˜54%), beet molasses (having a total sugar content of about 48˜52%), citrus molasses (having a total sugar content of about 40˜48%), and corn molasses (also known as starch molasses, and having a total sugar content of about 50%) based on the source thereof. Corn steep liquor is a by-product of the corn wet-milling industry, mainly contains about 47% protein, and can be used as nitrogen sources in media.

Since ingredients contained in molasses and corn steep liquor can be utilized by yeasts, media containing molasses and/or corn steep liquor can be used for cultivation of yeasts so as to increase the growth and fermentation yield of yeasts. For example, in Samuel Amartey and Thomas W. Jeffries (1994), Biotechnology letters., 16:211-214, Pichia stipitis CBS 6054 is cultivated in a medium containing only 30 g/L corn steep liquor to obtain an inoculum, followed by inoculating the inoculum into the same medium to conduct a fermentation reaction. The results reveal that, compared with other media, the growth rate of Pichia stipitis CBS 6054 cultivated in such medium is slightly increased, and the xylose utilization rate and the ethanol yield thereof are improved.

In order to mass produce β-glucan, which is the most plentiful polysaccharide in a cell wall, Kim Y. H. et al. are reported to have cultivated Saccharomyces cerevisiae JUL3 in a seed medium consisting of glucose, yeast extract, and peptone to obtain an inoculum, followed by inoculating the inoculum into media containing various concentrations of molasses and corn steep liquor for cultivation. The results reveal that the cell mass of Saccharomyces cerevisiae JUL3 is increased when it is cultivated in a medium containing 6.4% (v/v) molasses and 17% (v/v) corn steep liquor (Kim Y. H. et al. (2007), J. Ind. Eng. Chem., 13:153-158).

VU V. H. et al. are reported to have cultivated Saccharomyces cerevisiae KV-25 in a YPD medium to thus obtain an inoculum, followed by inoculating the inoculum into media containing various concentrations of molasses and corn steep liquor for cultivation. The results reveal that the optimized concentrations of molasses and corn steep liquor for high-cell-density cultivation are 10.25% (v/v) and 16.87% (v/v), respectively, and 36.5 g/L of cell mass can be obtained in a medium containing the optimized concentrations of molasses and corn steep liquor (VU V. H. and Kim K. (2009), J. Microbiol. Biotechnol., 19:1603-1611).

However, in the aforesaid prior art, a large amount of molasses and/or corn steep liquor is required during cultivation, so that cultivation costs may be increased, which would be unsuitable for industrial application. Additionally, the prior art simply discloses use of molasses and/or corn steep liquor as a nutrient source for yeast to increase the cell mass thereof. However, the effects of molasses and corn steep liquor on xylose utilization and ethanol yield of yeast in a cellulosic hydrolysate containing fermentation inhibitors (e.g., acetic acid) are not addressed.

### SUMMARY

Therefore, according to a first aspect, the disclosure provides a seed medium for the cultivation of a yeast cell capable of yielding ethanol by consumption of glucose and xylose, consisting essentially of molasses, corn steep liquor and water, wherein based on the total volume of the seed medium, the molasses has a concentration ranging from 1.0 to 6.0% (v/v), and the corn steep liquor has a concentration ranging from 4.0 to 8.0% (v/v), the concentration of the molasses being less than that of the corn steep liquor.

According to a second aspect, the disclosure provides a method for preparing a seed culture of a yeast cell, comprising cultivating a yeast cell capable of yielding ethanol to by consumption of glucose and xylose in the aforesaid seed medium so as to obtain the seed culture of the yeast cell in the seed medium.

According to a third aspect, the disclosure provides a method for producing ethanol from a biomass, comprising:

cultivating a yeast cell capable of yielding ethanol by consumption of glucose and xylose in the aforesaid seed medium so as to obtain a seed culture of the yeast cell in the seed medium; and

fermenting the biomass with the seed culture of the yeast cell;

wherein the biomass contains glucose, xylose, and acetic acid.

### BRIEF DESCRIPTION OF THE DRAWINGS

Other features and advantages of the disclosure will become apparent in the following detailed description of the embodiments with reference to the accompanying drawings, of which:

FIG. 1 is a bar diagram showing the ethanol yields of the double mutated Saccharomyces cerevisiae in Control Group 1 and Experimental Groups 1 to 5 after fermentation in a fermentation medium with the presence of acetic acid, before fermentation, the double mutated Saccharomyces cerevisiae in Control Group 1 being cultivated in a YPD medium, the double mutated Saccharomyces cerevisiae in each of Experimental Groups 1 to 5 being cultivated in a seed medium with 3% (v/v) of cane molasses and corn steep liquor at a respective concentration; and

FIG. 2 is a bar diagram showing the ethanol yields of the double mutated Saccharomyces cerevisiae in Control Group 2 and Experimental Groups 6 to 11 after fermentation in a fermentation medium with the presence of acetic acid, before fermentation, the double mutated Saccharomyces cerevisiae in Control Group 2 being cultivated in a YPD medium, the double mutated Saccharomyces cerevisiae in each of Experimental Groups 6 to 11 being cultivated in a seed medium with 6% (v/v) of corn steep liquor and cane molasses at a respective concentration.

### DETAILED DESCRIPTION

Cellulosic biomass contains a large amount of cellulose, hemicellulose, lignin, etc. which are intertwined to form a complex and rigid network structure. The network structure may cause limitations of the cellulosic biomass in production of ethanol. As such, the cellulosic biomass is usually subjected to an appropriate pretreatment, and then a degradation treatment using enzymes to hydrolyze the cellulosic biomass into reducing sugars such as, glucose, xylose, etc. However, after the above treatments are applied, fermentation inhibitors (e.g., acetic acid, furfural, hydroxymethyl furfural, etc.) would be generated, thereby adversely affecting the fermenting ability and ethanol yield of yeast cells.

In order to alleviate the undesired effects caused by the fermentation inhibitors so as to increase the xylose utilization rate and the ethanol yield, the Inventors endeavored to develop improved methods and found that cultivation of a glucose/xylose co-fermenting yeast cell in a seed medium containing molasses and corn steep liquor is effective in enhancing acetic acid resistance of the yeast cell at the subsequent fermentation stage. In other words, cultivation of the glucose/xylose co-fermenting yeast cell in the seed medium of the disclosure could improve xylose utilization rate of the seed culture of the glucose/xylose co-fermenting yeast cell under the fermentation condition where acetic acid is present, thereby resulting in an increase in ethanol yield.

In this disclosure, the seed medium for the cultivation of the yeast cell capable of yielding ethanol by consumption of glucose and xylose (i.e., the glucose/xylose co-fermenting yeast cell) consists essentially of molasses, corn steep liquor and water. Based on the total volume of the seed medium, the molasses has a concentration ranging from 1.0 to 6.0% (v/v), and the corn steep liquor has a concentration ranging from 4.0 to 8.0% (v/v). The concentration of the molasses is less than that of the corn steep liquor.

As used herein, the term “molasses” refers to syrup obtained by removing sucrose crystals from the massecuite during the refinement of sugars from a plant. The type of molasses suitable for use in the disclosure is not particularly limited, but may include various commercially available products. Preferably, the molasses is selected from the group consisting of cane molasses, beet molasses, citrus molasses, corn molasses, and combinations thereof. In an embodiment of the disclosure, the molasses is cane molasses.

In certain embodiments, based on the total volume of the seed medium, the concentration of the molasses ranges from 3.0 to 4.0% (v/v). In one embodiment of the disclosure, the concentration of the molasses is 3.0% (v/v).

As used herein, the term “corn steep liquor” refers to a concentrated liquid obtained by steeping of corn in diluted acid during a corn wet-milling process. Corn steep liquor suitable for the disclosure is not particularly limited, but may include various commercially available products.

In certain embodiments, based on the total volume of the seed medium, the concentration of the corn steep liquor ranges from 5.0 to 7.0% (v/v). In one embodiment of the disclosure, the concentration of the corn steep liquor is 6.Q (v/v).

As used herein, the term “water” includes, but is not limited to, deionized water, reverse osmosis water, distilled water, and double-distilled water (ddH₂O). In an embodiment of the disclosure, the water is deionized water.

The disclosure also provides a method for preparing a seed culture of the yeast cell, comprising cultivating the yeast cell in the aforesaid seed medium so as to obtain the seed culture of the yeast cell in the seed medium.

As used herein, the term “yeast cell” is intended to encompass all yeast strains capable of yielding ethanol by consumption of glucose and xylose, and the yeast cells suitable for use in the disclosure includes, but is not limited to, cells originated from Saccharomyces spp., Pichia spp., and Candida spp.

In an embodiment of the disclosure, the yeast cell is recombinant Saccharomyces cerevisiae. Preferably, the genomic DNA of the recombinant Saccharomyces cerevisiae includes a gene encoding xylose reductase (XR), a gene encoding xylulose kinase (XK) and a gene encoding xylitol dehydrogenase (XDH). More preferably, fps1 gene, which encodes glycerin passage protein, and gpd2 gene, which encodes glycerol 3-phosphate dehydrogenase-2 (GPD2), in the genomic DNA of the recombinant Saccharomyces cerevisiae are deleted, disrupted or disabled.

As used herein, the term “delete” refers to removing a full or partial coding region from a gene.

As used herein, the term “disrupt” refers to performing deletion, insertion or mutation of nucleotide(s) in a gene, so that the gene no longer produces an active enzyme, or produces an enzyme with severely reduced activity.

As used herein, the term “disable” refers to inactivating a gene or its encoded protein to thereby lose its original activity or function.

In an embodiment of the disclosure, the yeast cell is prepared by deleting or disrupting fps1 gene and gpd2 gene in the genomic DNA of a strain of Saccharomyces cerevisiae which is deposited in the Deutsche Sammlung von Mikroorganismen and Zellkulturen (DSMZ) under an accession number DSM 25508.

In another embodiment of the disclosure, the yeast cell is Pichia stipitis.

According to the disclosure, the cultivating step is conducted under an aerobic condition.

The disclosure also provides a method for producing ethanol from a biomass, comprising:

cultivating the yeast cell in the aforesaid seed medium so as to obtain the aforesaid seed culture of the yeast cell in the seed medium; and

fermenting the biomass with the seed culture of the yeast cell;

wherein the biomass contains glucose, xylose, and acetic acid.

According to the disclosure, the biomass is a mixed sugar solution comprising glucose, xylose, and acetic acid.

According to the disclosure, the biomass is a cellulosic hydrolysate comprising glucose, xylose, and acetic acid.

According to the disclosure, the cellulosic hydrolysate is prepared by performing a pretreatment and a hydrolysis treatment on a raw cellulosic biomass material in sequence.

As used herein, the term “cellulosic hydrolysate”, “lignocellulosic hydrolysate” and “biomass hydrolysate” can be used interchangeably, and refer to products generated from saccharification of biomass.

According to the disclosure, acetic acid in the biomass is present in an amount ranging from 4 to 10 g/L; preferably, from 5 to 8.5 g/L; more preferably, from 6 to 8 g/L.

According to the disclosure, the fermenting step is conducted under a condition that is substantially absent of molasses and corn steep liquor.

As used herein, the term “substantially absent of” refers to the lack of meaningful quantities of a specifically identified ingredient. Preferably, the fermenting step is conducted under a condition without the ingredient, or under a condition that the amount of the ingredient has no measurable effect on the fermenting step.

According to the disclosure, the fermenting step is conducted under a condition having a pH value ranging from 5.0 to 6.5; preferably, from 5.0 to 5.5.

According to the disclosure, the fermenting step is conducted under an anaerobic condition.

The disclosure will be further described by way of the following examples. However, it should be understood that the following examples are solely intended for the purpose of illustration and should not be construed as limiting the disclosure in practice.

### EXAMPLES:

Experimental Materials:

1. Δfps1Δgpd2 double mutant of Saccharomyces cerevisiae:

TheΔfps1Δgpd2 double mutant of Saccharomyces cerevisiae used in the following examples was prepared from Saccharomyces cerevisiae DSM 25508 in accordance with the procedures described in Zhang A et al. (2007), Letters in Applied Microbiology, 44:212-217 and Hubmann G. et al. (2011), Applied and Environmental Microbiology, 77:5857-5867. The method for producing Saccharomyces cerevisiae DSM 25508, which is capable of co-fermenting pentose and hexose, has been disclosed in US 20140087438 A1. In regard to the preparation of the Δfps1Δgpd2 double mutant of Saccharomyces cerevisiae, briefly speaking, fps1 gene of Saccharomyces cerevisiae DSM 25508 was deleted in accordance with the procedure described in Zhang A et al. (2007), supra, and the Δfps1 mutant strain thus obtained was then subjected to gpd2 gene deletion in accordance with the procedure described in Hubmann G. et al. (2011), supra, thereby obtaining the Δfps1Δgpd2 double mutant of Saccharomyces cerevisiae (referred to as “double mutated Saccharmyces cerevisiae” hereinafter).

2. Cane molasses and corn steep liquor for formulating a seed medium were respectively purchased from Fonen And FonHer Enterprise Co., LTD. and Taiwan Sugar Corporation. The cane molasses contains 435 g/L sucrose, 36.5 g/L glucose, and 86 g/L fructose, and the corn steep liquor contains 60.88% (w/w) of water, 17.67% (w/w) of crude proteins, 6.49% (w/w) of crude ashes, and 177.94 ppm of sulfur dioxide.

3. Glucose and xylose were purchased from Echo Chemical Co., LTD.

4. Urea was purchased from SHOWA.

5. Acetic acid was purchased from Scharlau.

6. The recipe of the YPD medium used in the examples is shown in Table 1.

TABLE 1

| Ingredients | Concentration (g/L) |
|---|---|
| Glucose | 40 |
| Yeast extract | 10 |
| Peptone | 20 |

The balance is deionized water.

7. The recipe of the fermentation medium for the double mutated Saccharomyces cerevisiae is shown in Table 2.

TABLE 2

| Ingredients | Concentration (g/L) |
|---|---|
| Glucose | 70 |
| Xylose | 50 |
| Urea | 1 |
| Acetic acid | 7 |

The balance is deionized water.

8. The recipe of the fermentation medium for Pichia stipitis ATCC 58376 (i.e., BCRC 21775) is shown in Table 3.

TABLE 3

| Ingredients | Concentration(g/L) |
|---|---|
| Xylose | 50 |
| Urea | 1 |
| Acetic acid | 7 |

The balance is deionized water.

General Experimental Procedure:

1. High performance liquid chromatography (HPLC)

The test samples were subjected to HPLC using a high performance liquid chromatograph (DIONEX Ultimate 3000) equipped with a refractive index detector (RI detector) according to the laboratory analytical procedures (LAPs) developed by National Renewable Energy Laboratory (NREL). The column and operation conditions for HPLC are as follows: Aminex HPX-87H column (BioRad); mobile phase: 5 mM sulfuric acid (in water); flow rate: 0.6 mL/min; sample injection volume: 20 μL; temperature of the column oven: 65° C.; and RI temperature: 45° C.

Furthermore, glucose (1.25-24 g/L), xylose (1.25-24 g/L), acetic acid (0.25-6 g/L), and ethanol (0,938-20 g/L) were used as control standards.

### Example 1

### Effect of Seed Medium (Containing 3% (v/v) of Cane Molasses and Corn Steep Liquor at a Respective Concentration) for Double Mutated Saccharomyces cerevisiae on Ethanol Yield

Experimental Procedures:

Six groups of the double mutated Saccharomyces cerevisiae (including a control group referred to as Control Group 1, and five experimental groups referred to as Experimental Groups 1 to 5) were provided. The double mutated Saccharomyces cerevisiae in Experimental Groups 1 to 5 were respectively inoculated into the seed media (10 mL) as shown in Table 4, and the double mutated Saccharomyces cerevisiae in Control Group 1 was inoculated into the YPD medium (10 mL) as shown in Table 1.

TABLE 4

Seed medium

| | | Concentration |
|---|---|---|
| Group | Ingredients | (%) (v/v) |
| Experimental Group 1 | cane molasses | 3 |
| | corn steep liquor | 4 |
| Experimental Group 2 | cane molasses | 3 |
| | corn steep liquor | 5 |
| Experimental Group 3 | cane molasses | 3 |
| | corn steep liquor | 6 |
| Experimental Group 4 | cane molasses | 3 |
| | corn steep liquor | 7 |
| Experimental Group 5 | cane molasses | 3 |
| | corn steep liquor | 8 |

The balance is deionized water.

Subsequently, each group was incubated in a thermo shaker incubator (30° C., 200 rpm) for 16 hours, followed by going through centrifugation at 15,700 g for 1 minute to obtain a cell precipitate as a seed culture of the double mutated Saccharomyces cerevisiae.

Then, the seed culture of each group was inoculated in 100 mL of the fermentation medium as shown in Table 2 at a concentration of 2×10⁶ cell/mL, followed by fermentation in a thermo shaker incubator (30° C., 200 rpm) under anaerobic condition for 72 hours. For each group, at 6 hours and 24 hours after the initiation of fermentation, 6N NaOH was added to maintain the pH value of each group at 5.5.

Afterwards, the fermentation culture of each group was subjected to centrifugation at 12,000 rpm for 1 minute to obtain a supernatant, which is then conducted with HPLC along the lines as described in the previous “General Experimental Procedure” section to determine the ethanol content in the supernatant.

Ethanol yield was calculated using the following Equation (I):

A=[B/(C×0.51+D×0.48)]×100 (I)

where A=ethanol yield (%)

B=ethanol content detected in the supernatant (g/L)

C=glucose content in the fermentation medium before fermentation (g/L)

D=xylose content in the fermentation medium before fermentation (g/L)

Results:

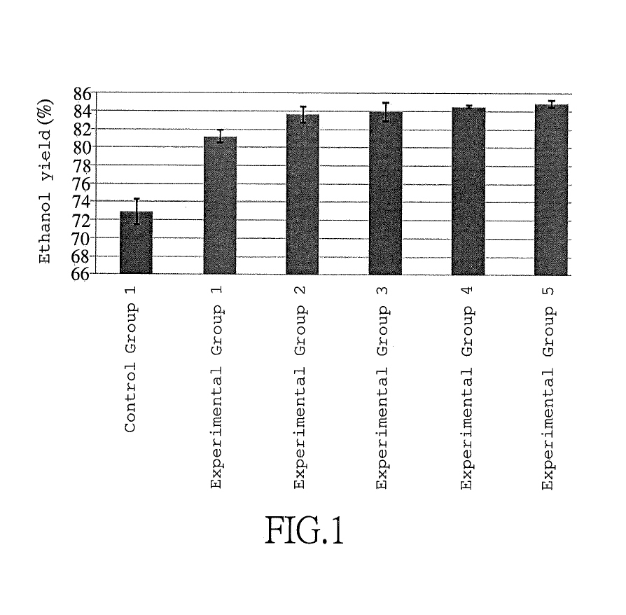

The ethanol yields of Control Group 1 and Experimental Groups 1 to 5 are shown in FIG. 1. It can be seen from FIG. 1 that the ethanol yield of each Experimental Group is significantly higher as compared with Control Group 1, and increases with an increase of the concentration of the corn steep liquor. The experimental results reveal that the seed medium containing 3% (v/v) of cane molasses and corn steep liquor at a respective concentration is effective in enhancing the ethanol yield in the fermentation medium with acetic acid.

### Example 2

### Effect of Seed Medium (Containing 6% (v/v) of Corn Steep Liquor and Cane Molasses at a Respective Concentration) for Double Mutated Saccharomyces cerevisiae on Ethanol Yield

Experimental Procedures:

Seven groups of the double mutated Saccharomyces cerevisiae (including a control group referred to as Control Group 2, and six experimental groups referred to as Experimental Groups 6 to 11) were provided. The double mutated Saccharomyces cerevisiae in Experimental Groups 1 to 6 were respectively inoculated into the seed media (10 mL) as shown in Table 5, and the double mutated Saccharomyces cerevisiae in Control Group 2 was inoculated into the YPD medium (10 mL) as shown in Table 1.

TABLE 5

Seed medium

| | | Concentration |
|---|---|---|
| Group | Ingredients | (%) (v/v) |
| Experimental Group 6 | cane molasses | 1 |
| | corn steep liquor | 6 |
| Experimental Group 7 | cane molasses | 2 |
| | corn steep liquor | 6 |
| Experimental Group 8 | cane molasses | 3 |
| | corn steep liquor | 6 |
| Experimental Group 9 | cane molasses | 4 |
| | corn steep liquor | 6 |
| Experimental Group 10 | cane molasses | 5 |
| | corn steep liquor | 6 |
| Experimental Group 11 | cane molasses | 6 |
| | corn steep liquor | 6 |

The balance is deionized water.

Subsequently, each group was incubated in a thermo shaker incubator (30°C, 200 rpm) for 16 hours, followed by going through centrifugation at 15,700 g for 1 minute to obtain a cell precipitate as a seed culture of the double mutated Saccharomyces cerevisiae.

Then, the seed culture of each group was inoculated in 100 mL of the fermentation medium as shown in Table 2 at a concentration of 2×10⁶ cell/mL, followed by fermentation in an thermo shaker incubator (30° C., 200 rpm) under anaerobic condition for 72 hours. For each group, at 6 hours and 24 hours after the initiation of fermentation, 6N NaOH was added to maintain the pH value of each group at 5.5.

Afterwards, the fermentation culture of each group was subjected to centrifugation at 12,000 rpm for 1 minute to obtain a supernatant, followed by conducting HPLC along the lines as described in the previous section of “General Experimental Procedure” to determine the ethanol content in the supernatant. Ethanol yield was to calculated using Equation (I) described in Example 1.

Results:

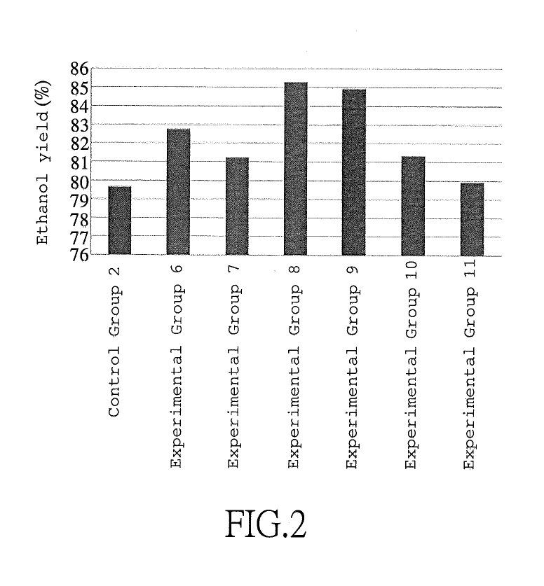

The ethanol yields of Control Group 2 and Experimental Groups 6 to 11 are shown in FIG. 2. It can be seen from FIG. 2 that the ethanol yield of each Experimental group is higher compared to Control Group 2. In particular, Experimental Group 8 exhibits the highest ethanol yield. The experimental results reveal that the seed medium containing 6% (v/v) of corn steep liquor and cane molasses at a respective concentration, especially, the seed medium containing 6% (v/v) of corn steep liquor and 3% (v/v) of cane molasses, is effective in enhancing the ethanol yield in the fermentation medium with acetic acid.

### Example 3

### Effect of Seed Medium (Containing 3% (v/v) of Cane Molasses and 6% (v/v) of Corn Steep Liquor) on Ethanol Yield and Glucose and Xylose Consumption in Fermentation Media with Various pH Values

In this example, the fermentation media containing acetic acid and respectively having a pH value of 5.0, 5.5 and 6.0 were used to mimic a biomass containing acetic acid (e.g. hydrolyzed cellulosic biomass), and the effect of the seed medium containing 3% (v/v) of cane molasses and 6% (v/v) of corn steep liquor on the glucose and xylose utilization rates and ethanol yield of the double mutated Saccharomyces cerevisiae in the fermentation medium was evaluated.

Experimental Procedures:

Six groups of the double mutated Saccharomyces cerevisiae (including three control groups referred to as Control Groups 3 to 5, and three experimental groups referred to as Experimental Groups 12 to 14) were provided. The double mutated Saccharomyces cerevisiae in each of Experimental Groups 12 to 14 was inoculated into a seed medium (10 mL) containing 3% (v/v) of cane molasses and 6% (v/v) of corn steep liquor. The double mutated Saccharomyces cerevisiae in each of Control Groups 3 to 5 was inoculated into the YPD medium (10 mL) as shown in Table 1.

Subsequently, each group was incubated in a thermo shaker incubator (30° C., 200 rpm) for 16 hours, followed by going through centrifugation at 15,700 g for 1 minute to obtain a cell precipitate.

Then, the cell precipitate of each group was inoculated in 100 mL of the fermentation medium as shown in Table 2 at a concentration of 2×10⁶ cell/mL, followed by fermentation in a thermo shaker incubator (30° C., 200 rpm) under anaerobic condition for 72 hours. For each group, at 6 hours and 24 hours after the initiation of fermentation, 6N NaOH was added to maintain the pH values of Experimental Group 12 and Control Group 3 at 5, the pH values of Experimental Group 13 and Control Group 4 at 5.5, and the pH values of Experimental Group 14 and Control Group 5 at 6.0.

Afterwards, the fermentation culture of each group was subjected to centrifugation at 12,000 rpm for 1 minute to obtain a supernatant, followed by conducting HPLC along the lines as described in the previous section of “General Experimental Procedure” to determine the ethanol content, the xylose content, and the glucose content in the supernatant. Ethanol yield was calculated using Equation (I) described in Example 1.

Results:

The ethanol yield, the xylose content, and the glucose content of each of Control Groups 3 to 5 and Experimental Groups 12 to 14 are shown in Table 6.

TABLE 6

| | pH value | Ethanol | Glucose | Xylose |
|---|---|---|---|---|
| | of fermentation | yield | content | content |
| Group | medium | (%) | (g/L) | (g/L) |
| Experimental | 5.0 | 70.8 | 0 | 21.3 |
| Group 12 | | | | |
| Control | | 63.4 | 0 | 33 |
| Group 3 | | | | |
| Experimental | 5.5 | 83.9 | 0 | 3 |
| Group 13 | | | | |
| Control | | 72.9 | 0 | 10 |
| Group 4 | | | | |
| Experimental | 6.0 | 84.8 | 0 | 0 |
| Group 14 | | | | |
| Control | | 84.1 | 0 | 3.9 |
| Group 5 | | | | |

It can be seen from Table 6 that the glucose content detected in each group is 0 g/L no matter what the seed medium is and no matter what the pH value of the fermentation medium is. In addition, the ethanol yield of each Experimental Group is higher than that of the corresponding Control Group, and the xylose content detected in each Experimental Group is lower than that of the corresponding Control Group. It is noted that the ethanol yield of Experimental Group 12 at pH 5.0 is close to that of Control Group 4 at pH 5.5, and the ethanol yield of Experimental Group 13 at pH 5.5 is close to that of Control Group 5 at pH 6.0. The experimental results reveal that the seed media containing 3% (v/v) of cane molasses and 6% (v/v) of corn steep liquor is effective in enhancing the xylose utilization, thereby enhancing the ethanol yield even in a lower pH environment. The results indicate that even when the pH value of the fermentation medium is decreased with time during fermentation, the ethanol yield and xylose utilization may still be maintained at desired levels by virtue of cultivation of the double mutated Saccharomyces cerevisiae in the seed medium before fermentation. Therefore, the amount of an alkaline agent (e.g., NaOH) used for adjusting the pH value of the fermentation medium may be reduced.

### Example 4

### Effect of Seed Medium (Containing 3% (v/v) of Cane Molasses and 6% (v/v) of Corn Steep Liquor) on Ethanol Yield of Pichia stipitis ATCC 58376 in Fermentation Medium

In this example, Pichia stipitis ATCC 58376 (corresponding to BCRC 21775), which was purchased from the Biosource Collection and Research Center (BCRC) of the Food Industry Research and Development Institute (FIRDI), was used as a tested strain, and the fermentation medium containing acetic acid and having a pH value of 6.0 or 6.5 was used to mimic a biomass containing acetic acid (e.g. hydrolyzed cellulosic biomass).

Experimental Procedures:

Four groups of Pichia stipitis ATCC 58376 (including two control groups referred to as Control Groups 6 and 7, and two experimental groups referred to as Experimental Groups 15 and 16) were provided. The Pichia stipitis ATCC 58376 in each of Experimental Groups 15 and 16 was inoculated into a seed medium (10 mL) containing 3% (v/v) of cane molasses and 6% (v/v) of corn steep liquor. The Pichia stipitis ATCC 58376 in each of Control Groups 6 and 7 was inoculated into the YPD medium (10 mL) as shown in Table 1.

Subsequently, each group was incubated in a thermo shaker incubator (30° C., 200 rpm) for 16 hours, followed by going through centrifugation at 15,700 g for 1 minute to obtain a cell precipitate.

Then, the cell precipitate of each group was inoculated in 100 mL of the fermentation medium as shown in Table 3 at a concentration of 2×10⁶ cell/mL, followed by fermentation in an thermo shaker incubator (30° C., 200 rpm) under anaerobic condition for 72 hours. For each group, at 6 hours and 24 hours after the initiation of fermentation, 6N NaOH was added to maintain the pH value of Experimental Group 15 and Control Group 6 at 6.0 and the pH value of Experimental Group 16 and Control Group 7 at 6.5.

Afterwards, the fermentation culture of each group was subjected to centrifugation at 12,000 rpm for 1 minute to obtain a supernatant, followed by conducting HPLC along the lines as described in the previous section of “General Experimental Procedure” to determine the ethanol content in the supernatant.

Ethanol yield was calculated using the following Equation (II):

E=[F/(G×0.48)]×100 (II)

where E=ethanol yield (%)

F=ethanol content detected in the supernatant (g/L)

G=xylose content in the fermentation medium before fermentation (g/L)

Results:

The ethanol yields of Control Groups 6 and 7 and Experimental Groups 15 and 16 are shown in Table 7.

TABLE 7

| | pH value of | |
|---|---|---|
| | fermentation | Ethanol yield |
| Group | medium | (%) |
| Experimental Group 15 | 6.0 | 18.8 |
| Control Group 6 | | 8.1 |
| Experimental Group 16 | 6.5 | 17.9 |
| Control Group 7 | | 16.3 |

It can be seen from Table 7 that the ethanol yield of each Experimental Group is higher than that of the corresponding Control Group. Particularly, the ethanol yield of Experimental Group 15 is close to that of Control Group 7. The experimental results reveal that the seed medium containing 3% (v/v) of cane molasses and 6% (v/v) of corn steep liquor is effective in enhancing the ethanol yield in the fermentation medium with acetic acid at pH 6.0 and 6.5. The results indicate that even when the pH value of the fermentation medium is decreased with time during fermentation, the ethanol yield may still be maintained at a desired level by virtue of cultivation of the Pichia stipitis ATCC 58376 in the seed medium before fermentation. Therefore, the amount of an alkaline agent (e.g., NaOH) used for adjusting the pH value of the fermentation medium may be reduced.

In view of the foregoing, cultivation of yeast capable of co-fermenting glucose and xylose in seed medium containing cane molasses and corn steep liquor is effective in enhancing the xylose utilization, thereby enhancing the ethanol yield in the fermentation medium with acetic acid.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

1. A seed medium for the cultivation of a yeast cell capable of yielding ethanol by consumption of glucose and xylose, consisting essentially of molasses, corn steep liquor and water,
wherein, based on the total volume of said seed medium, said molasses has a concentration ranging from 1.0 to 6.0% (v/v), and said corn steep liquor has a concentration ranging from 4.0 to 8.0% (v/v), the concentration of said molasses being less than that of said corn steep liquor.

2. The seed medium of claim 1, wherein, based on the total volume of said seed medium, the concentration of said molasses ranges from 3.0 to 4.0% (v/v).

3. The seed medium of claim 1, wherein, based on the total volume of said seed medium, the concentration of said corn steep liquor ranges from 5.0 to 7.0% (v/v).

4. The seed medium of claim 1, wherein said molasses is selected from the group consisting of cane molasses, beet molasses, citrus molasses, corn molasses, and combinations thereof.

5. A method for preparing a seed culture of a yeast cell, comprising cultivating a yeast cell capable of yielding ethanol by consumption of glucose and xylose in a seed medium as claimed in claim 1 so as to obtain the seed culture of the yeast cell in the seed medium.

6. The method of claim 5, wherein the yeast cell is selected from the group consisting of recombinant Saccharomyces cerevisiae and pichia stipitis.

7. The method of claim 6, wherein the genomic DNA of the recombinant Saccharomyces cerevisiae includes a gene encoding xylose reductase, a gene encoding xylulokinase and a gene encoding xylitol dehydrogenase.

8. method of claim 6, wherein fps1 gene and gpd2 gene in the genomic DNA of the recombinant Saccharomyces cerevisiae are deleted, disrupted or disabled.

9. The method of claim 5, wherein the cultivating step is conducted under an aerobic condition.

10. A method for producing ethanol from a biomass, comprising:
cultivating a yeast cell capable of yielding ethanol by consumption of glucose and xylose in a seed medium as claimed in claim 1 so as to obtain a seed culture of the yeast cell in the seed medium; and
fermenting the biomass with the seed culture of the yeast cell;
wherein the biomass contains glucose, xylose, and acetic acid.

11. The method of claim 10, wherein the yeast cell is selected from the group consisting of recombinant Saccharomyces cerevisiae and pichia stipitis.

12. The method of claim 11, wherein the genomic DNA of the recombinant Saccharomyces cerevisiae includes a gene encoding xylose reductase, a gene encoding xylulokinase and a gene encoding xylitol dehydrogenase.

13. The method of claim 11, wherein fps1 gene and gpd2 gene in the genomic DNA of the recombinant Saccharomyces cerevisiae are deleted, disrupted or disabled.

14. The method of claim 10, wherein the cultivating step is conducted under an aerobic condition.

15. The method of claim 10, wherein the fermenting step is conducted under a condition that is substantially absent of molasses and corn steep liquor.

16. The method of claim 10, wherein the fermenting step is conducted under a condition having a pH value ranging from 5.0 to 6.5.

17. The method of claim 10, wherein the fermenting step is conducted under an anaerobic condition.

18. The method of claim 10, wherein acetic acid in the biomass is present in an amount ranging from 4 to 10 g/L.

19. The method of claim 10, wherein the biomass is a cellulosic hydrolysate.
